(19) United States
(12) Patent Application Publication
(10) Pub. No.: US 2022/0054854 A1
(43) Pub. Date: Feb. 24, 2022

(54) PHOTOTHERAPEUTIC GLASSES

(71) Applicant: WENZHOU TIDE-OPTICAL CO., LTD., Wenzhou City (CN)

(72) Inventor: Leping ZHENG, Wenzhou City (CN)

(21) Appl. No.: 17/011,757

(22) Filed: Sep. 3, 2020

(30) Foreign Application Priority Data
Aug. 18, 2020 (CN) 202021724957.8

(51) Int. Cl. A61N 5/06

(52) U.S. Cl. CPC A61N 5/06; A61N 2005/0648; A61N 2005/0663; A61N 2005/0652; A61N 2005/0632

(57) ABSTRACT

A phototherapeutic pair of glasses comprising a glasses frame, having a front frame and a pair of glasses legs pivotally connected to a corresponding transverse outer end of the front frame. A plurality of groups of a first physiotherapy LED lamp group are arranged on one side, facing the human face, of the front frame, and the first physiotherapy LED lamp group is distributed along the circumference of an eye. The physiotherapy LED lamp group is arranged on one side, facing the human face, of the front frame of the glasses. The physiotherapy LED lamp group irradiates the periphery of the eye to perform phototherapy on the periphery of the eye during the wearing process such that a beauty effect is achieved. Meanwhile, the wearer is not influenced, and the beauty enjoyment brought by phototherapy can be enjoyed in the entertainment process of watching television and the like.

### PRIORITY CLAIM

The present application claims priority to Chinese Patent Application No. 202021724957.8, filed on Aug. 18, 2020, which said application is incorporated by reference in its entirety herein.

### TECHNICAL FIELD

The invention relates to the field of eyeglasses, in particular to phototherapeutic eyeglasses.

### BACKGROUND

Common eyeglasses (“glasses”) are composed of glasses frames and lenses, and according to different lenses, the glasses can have the functions of correcting vision, shading sunlight, and beautifying, in addition to any health-care function. People usually use special instruments and equipment to protect their eyes to achieve the effects of eye care, protecting eyes and beautifying eyes.

However, since glasses are necessary tools for a myopic person, when the eyes are being cared for, the glasses are generally required to be removed. When the glasses are removed, the entertainment requirements of the myopic person, such as watching television, reading books and other entertainment activities, are greatly influenced. So combining eye care and glasses can become an effective way to utilize the time utilization when the eyes are cared for.

### SUMMARY

Aiming at the defects of the prior art, the invention aims to provide a phototherapeutic pair of glasses.

In order to achieve the above object, the invention provides the following technical solution:

a phototherapeutic pair of glasses, comprising a glasses frame, wherein the glasses frame comprises a front frame and a pair of glasses legs pivotally connected to a corresponding transverse outer end of the front frame, a plurality of groups of a first physiotherapy LED lamp group is arranged on one side, facing the human face, of the front frame, and the first physiotherapy LED lamp group is distributed along the circumference of an eye.

The bottom of the front frame extends downwards to form a mounting part, and the first physiotherapy LED lamp group is arranged on the mounting part.

A lens mounting groove is formed in the front frame, and a light blocking strip is arranged between the first physiotherapy LED lamp group and the lens mounting groove.

A vibrator is arranged in the light blocking strip.

The light blocking strip is made of a flexible material.

A vibrator is arranged in the light blocking strip.

A first mounting cavity is arranged in the mounting part and the first physiotherapy LED lamp group is arranged in the first mounting cavity. A plurality of through holes are formed in one side, facing the human face, of the first mounting cavity, and the first physiotherapy LED lamp group and the through holes are arranged in one-to-one correspondence, i.e., one through hole for each LED.

The first physiotherapy LED lamp group is set to be two groups.

Any first physiotherapy LED lamp group comprises a plurality of LEDs, and the LEDs are arranged along the periocular direction.

The LEDs are arranged in an eye bag pattern direction.

The LED comprises three luminous tubes with different colors.

The LED comprises a red light luminous tube, an orange light luminous tube and a blue light luminous tube.

A second physiotherapy LED lamp group is arranged on one side, facing the human face, of the front end of the glasses leg, and the second physiotherapy LED lamp group is arranged along the direction of fishtail lines (facial lines or wrinkles at, adjacent to, or around an eye).

The second physiotherapy LED lamp group comprises at least two rows of LEDs.

A second mounting cavity is arranged at the front end of the glasses leg, and the second physiotherapy LED lamp group is configured to be fixedly arranged in the second mounting cavity.

A battery-containing cavity is arranged at the rear end of the second mounting cavity, and a battery is arranged in the battery containing cavity.

The battery is a rechargeable battery, and a charging interface is arranged on any side of the battery containing cavity, and the charging interface is electrically connected with the rechargeable battery.

A control switch capable of controlling the working state of the first physiotherapy LED lamp group is arranged on any one side of the glasses leg.

A control switch capable of controlling the working state of the first physiotherapy LED lamp group and the second physiotherapy LED lamp group is arranged on any one side of the glasses leg.

The invention has beneficial effects as follows. The physiotherapy LED lamp group is arranged on one side, facing the human face, of the front frame of the glasses. The physiotherapy LED lamp group is used for irradiating the periphery of the eye to perform phototherapy on the periphery of the eye in the wearing process such that a beauty effect is achieved. Meanwhile, the wearer is not influenced, and the beauty enjoyment brought by phototherapy can be enjoyed in the entertainment process of watching television and the like.

### BRIEF DESCRIPTION OF THE DRAWINGS

FIG. 1 is a schematic view showing the structure of the present invention, according to an embodiment.

FIG. 2 is a side view of a glasses temple of the present invention, according to an embodiment.

FIG. 3 is a schematic plan view of the physiotherapy LED lamp of the present invention, according to an embodiment.

FIG. 4 is a schematic circuit diagram of the physiotherapy LED lamp group of the present invention, according to an embodiment.

### DETAILED DESCRIPTION OF THE INVENTION

The technical solutions in the embodiments of the present invention will be clearly and completely described below in conjunction with the accompanying drawings in the embodiments of the present invention. Obviously, the described embodiments are only a part of the embodiments of the present invention, rather than all the embodiments. Based on the embodiments of the present invention, all other embodiments obtained by one of ordinary skill in the art without involving any inventive effort are within the scope of the present invention.

It should be noted that all directional indicators (such as up, down, left, right, front, rear . . . ) in embodiments of the present invention are used only to explain the relative positional relationships, motion conditions, etc. between components in a particular pose (as shown in the drawings), which changes accordingly if the particular pose changes.

In the present invention, the terms “connected, secured”, and the like are to be construed broadly, unless expressly stated and defined otherwise, e.g., “secured” may be a fixed connection, or may be detachably connected, or integrated; can be a mechanical connection or an electrical connection; may be joined directly or indirectly through intervening media; may be either internal connection between two elements, or an interaction of two elements, unless expressly defined otherwise. The specific meaning of the above terms in the present invention will be understood by those of ordinary skill in the art, as the case may be.

In addition, descriptions such as those relating to “first, second”, etc. in the present invention are used for descriptive purposes only and are not to be construed as indicating or implying relative importance or implicitly indicating the number of technical features indicated.

Therefore, features defining “first” and “second” may explicitly or implicitly include at least one such feature. In addition, the technical solutions of various embodiments can be combined with each other, but must be realized by a person skilled in the art. When the combination of the technical solutions contradicts each other or cannot be realized, the combination of the technical solutions should neither be considered to exist nor be within the scope of protection of the present invention.

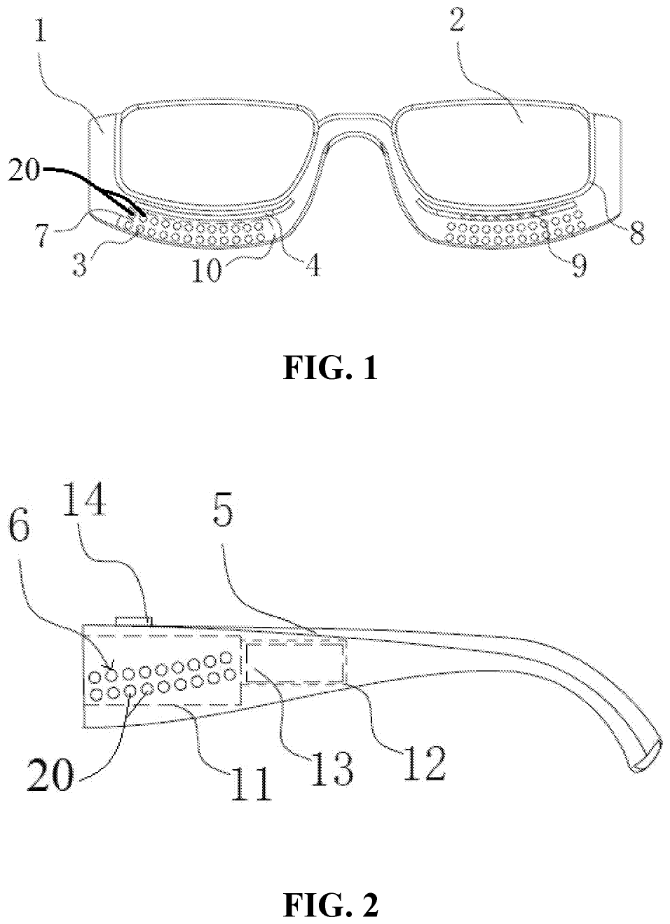

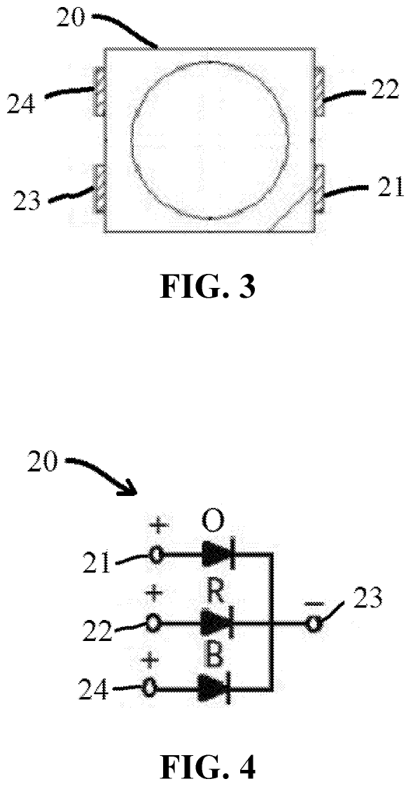

By the circumference of an eye is meant around an eye. Referring to FIGS. 1 and 2, the invention discloses a phototherapeutic pair of glasses, comprising a glasses frame, wherein the glasses frame comprises a front frame 1 and a pair of glasses legs 5 pivotally connected to a corresponding transverse outer end of the front frame. The front frame 1 can be a full frame or a half frame, and the half frame is in the form of a half frame with an exposed upper side and a connected lower side, and the side of the front frame 1 which faces a human face is provided with a plurality of a first physiotherapy LED lamp group 3, which includes a plurality of LED lamps 20 (see also, FIGS. 3-4). The first physiotherapy LED lamp group 3 is distributed along the circumference of an eye, that is, the first physiotherapy LED lamp group 3 is arranged in a mode of covering the circumference range of an eye so that the LED lamps 20 can cover or light up the periphery of the eye, including the facial area around the eye, such as the position where a fishtail line, an eye bag and/or a dark circle are located, and the beauty effect is realized by alternately changing the wavelength and the color of the LEDs. The bottom of the front frame extends downwards to form a mounting part 7, wherein the bottom of the front frame is widened, the width of the front frame meets the area of covering a dark circle or an eye bag on a face (underneath the eye), and the first physiotherapy LED lamp group 3 is arranged on the mounting part.

The front frame is provided with a lens mounting groove 8, wherein the lens 2 is configured to be arranged in the lens mounting groove 8 and a light blocking strip 4 is arranged between the first physiotherapy LED lamp group 3 and the lens mounting groove 8. The length of the light blocking strip 4 is matched with the length of the first physiotherapy LED lamp group so that the light of the first physiotherapy LED lamp group is prevented from entering human eyes in the wearing process. Concerning the thickness of the light blocking strip when the glasses are worn, the surface of the light blocking strip is preferably in contact with the human face skin such that the isolation effect is optimal.

The light blocking strip 4 is made of a flexible material such that the human body feels more comfortable when the light blocking strip is worn, and the flexible material may be made of silica gel and the like.

Furthermore, in an embodiment, a vibrator 9 is arranged in the light blocking strip, and by arranging the vibrator 9 in the light blocking strip, the face can be massaged to promote blood circulation.

A first mounting cavity 10 is arranged in the mounting part 7. The first mounting cavity 10 is arranged in the mounting part such that a circuit board with the first physiotherapy LED lamp group is arranged in the first mounting cavity 10 to prevent the circuit board from being directly exposed and influencing the perceived beauty of the glasses and wearer of the glasses. A plurality of through holes is formed on one side of the mounting part 7, facing the human face, and in an embodiment, the first physiotherapy LED lamp group 6 and the through holes are arranged in a one-to-one correspondence mode, one LED lamp 20 for each through hole, so that light rays of the LED lamps 20 of the LED lamp group 3 are guided out through the through holes. Or a light guide column can be arranged in the through holes so that full coverage of a light source is achieved through the cooperation among the light guide columns. Meanwhile, the light guide columns play a role of sealing the through holes such that secretion blocking the through holes caused when the human face directly contacts with the through holes can be avoided and the phototherapeutic effect is prevented from being influenced.

The first physiotherapy LED lamp group 3 is set to be two groups distributed up and down and arranged around the edge of the lens. The first physiotherapy LED lamp group 3 covers the peripheral range of the eye and facial area around the eye, and is preferably arranged only on the lower side of the front frame to cover the lower position of the eye, or the lower periocular portion of the eye.

In an embodiment, a first physiotherapy LED lamp group 3 comprises a plurality of LED lamps 20, and the LEDs are arranged along a periocular direction, or circumferentially about the eye.

The first physiotherapy LED lamp group 3 is arranged on the lower side of the front frame, that is, the LED lamps 20 are arranged in an eye bag pattern direction and cover the position of the lower side of a human eye for eliminating eye bag patterns or dark circles. A second physiotherapy LED lamp group 6 is arranged on one side, facing the human face, of the front end of the glasses leg 5, and the second physiotherapy LED lamp group 6 is arranged along the direction of the fishtail lines, or from a front of a human head to a back of the human head.

In an embodiment, the second physiotherapy LED lamp group 6 comprises at least two rows of LED lamps 20, wherein the LED lamps 20 are arranged in an upper layer and a lower layer or row, and the full-range coverage is realized, and the beauty effect is improved. In another embodiment, the LED lamp group 6 may comprise one group of LED lamps 20.

A second mounting cavity 11 is arranged at the front end of the glasses leg 5, and the second physiotherapy LED lamp group 6 is configured to be fixedly arranged in the second mounting cavity 11. The front end of the glasses leg 5 is provided with a second mounting cavity 11, wherein the second physiotherapy LED lamp group is fixedly arranged in the second mounting cavity 11 through a circuit board by a cover plate. The LEDs of the second physiotherapy LED lamp group 6 are exposed through a through hole in the cover plate or transmit light beams through the through hole or a light guide column so that the second physiotherapy LED lamp group 6 can comprehensively cover the position where the fishtail lines are located, and the phototherapy effect is improved.

Referring to FIGS. 3 and 4, the physiotherapy LED lamp group 3 or 6 comprises a plurality of LED lamps 20, wherein each LED lamp 20 is a single structure formed by packaging three light-emitting diodes, each with a different color, such as a red light LED, orange light LED and blue light LED. The wavelength of the red light is 635-645 nm, the wavelength of the blue light is 445-450 nm, and the wavelength of the orange light is 600-610 nm. A common cathode connection method is adopted such that the independent control of light with different colors can be realized and the switching of light with different wavelengths can be realized. As depicted, LED lamp 20 may include the common cathode 23, and individual anodes 21, 22 and 24.

Referring again to FIG. 2, a battery containing cavity 12 is arranged at the rear end of the second mounting cavity 11, wherein a battery 13 is arranged in the battery containing cavity 12, and the battery containing cavity 12 is in communication with the second mounting cavity 11, and the two containing cavities can be covered by using the same cover plate, and a sealing structure such as a sealing ring and the like is arranged between the cover plate and the two cavities so that the tightness of the cavities is realized. The battery 13, in an embodiment, is a rechargeable battery, and a charging interface is arranged on any side of the battery containing cavity 12. The charging interface is electrically connected with the rechargeable battery 13, and the charging interface is connected with a charging control panel connected with the rechargeable battery 13 so that the battery can be recycled.

A control switch 14 capable of controlling the working state of the first physiotherapy LED lamp group is arranged on any one side of the glasses leg.

A control switch 14 capable of controlling the working state of the first physiotherapy LED lamp group and the second physiotherapy LED lamp group is arranged on any one side of the glasses leg.

The control switch 14 can be a push switch, a touch switch and the like, can also be realized according to settings, and can be controlled in multiple functions by a single control switch 14, such as long press to realize light switching, illumination time adjustment of different wavelengths and the like.

The embodiments should not be construed as limiting the invention, and any modifications based on the spirit of the invention are intended to be within the scope of the invention.

1. A phototherapeutic pair of glasses, comprising:
a glasses frame, the glasses frame including a front frame and a pair of glasses legs pivotally connected to a corresponding transverse outer end of the front frame, the front frame defining two rows of front through holes facing a first direction, at least one of the glasses legs defining side through holes facing a second direction, the side through holes of the glasses legs facing a second direction that is transverse to the first direction, each of the first and second through holes forming light guide columns,
a lens mounted to the glasses frame,
a light blocking strip located between the lens and the two rows of through holes,
a first plurality of light emitting diodes (LEDs) of a first physiotherapy LED lamp group is arranged such that each LED of the plurality of LEDs is within one of the through holes of the front frame, and the first physiotherapy LED lamp group is distributed in a periocular direction along the circumference of an eye such that each of the two rows of through holes defines an arcuate path, and light from the first plurality of LEDs is guided through each respective light-guide column.

2. The phototherapeutic pair of glasses of claim 1, wherein a bottom of the front frame extends downwards to form a mounting part, and the first physiotherapy LED lamp group is arranged on the mounting part.

3. The phototherapeutic pair of glasses of claim 1, wherein the lens is located in a lens mounting groove that is formed in the front frame.

4. The phototherapeutic pair of glass of claim 3, further comprising a vibrator arranged in the light blocking strip.

5. The phototherapeutic pair of glasses of claim 1, wherein the light blocking strip is made of a flexible material.

6. The phototherapeutic pair of glasses of claim 5, further comprising a vibrator arranged in the light blocking strip.

7. The phototherapeutic pair of glasses of claim 1, wherein the mounting part defines a first mounting cavity , the first physiotherapy LED lamp group is arranged in the first mounting cavity, a first plurality of through holes are in communication with the first mounting cavity and are formed on one side of the mounting part, facing the human face, and the first physiotherapy LED lamp group and the through holes are arranged in one-to-one correspondence, such that the first mounting cavity defines one through hole for each LED lamp of the first physiotherapy LED lamp group.

8. The phototherapeutic pair of glasses of claim 1, wherein the first physiotherapy LED lamp group comprises two groups of LEDs, one group arranged in one of the two rows and the other group arranged in the other of the two rows.

9. (canceled)

10. The phototherapeutic pair of glasses of claim 9, wherein the LEDs are arranged in an eye bag pattern direction.

11. The phototherapeutic pair of glasses of claim 9, wherein each LED lamp is composed of three light-emitting diodes, each with a different color.

12. The phototherapeutic pair of glasses of claim 11, wherein each LED comprises a red light luminous tube, an orange light luminous tube and a blue light luminous tube.

13. The phototherapeutic pair of glasses of claim 1, further comprising a second physiotherapy LED lamp group arranged on one side in the second through holes, configured to face the human face, of the front end of the glasses leg, and the second physiotherapy LED lamp group is arranged lengthwise along the glasses leg so as to be arranged along a direction of a fishtail lines of a human face when the glasses are placed on a face of a human.

14. The phototherapeutic pair of glasses of claim 13, wherein the second physiotherapy LED lamp group comprises at least two rows of LEDs.

15. The phototherapeutic pair of glasses of claim 13, wherein the front end of the glasses leg is provided with a second mounting cavity, and the second physiotherapy LED lamp group is configured to be fixedly arranged in the second mounting cavity.

16. The phototherapeutic pair of glasses of claim 15, wherein a battery containing cavity is arranged at a rear end of the second mounting cavity, and a battery is arranged in the battery containing cavity.

17. The phototherapeutic pair of glasses of claim 16, wherein the battery is a rechargeable battery, a charging interface is arranged on a side of the battery containing cavity, and the charging interface is electrically connected with the rechargeable battery.

18. The phototherapeutic pair of glasses of claim 1, further comprising a control switch capable of controlling the working state of the first physiotherapy LED lamp group, the control switch arranged on one glasses leg.

19. The phototherapeutic pair of glasses of claim 13, further comprising a control switch capable of controlling the working state of the first physiotherapy LED lamp group and the second physiotherapy LED lamp group, the control switch is arranged on one side of the glasses leg.
